(19) United States
(12) Patent Application Publication
(10) Pub. No.: US 2014/0107265 A1
(43) Pub. Date: Apr. 17, 2014

(54) FACILITATED TRANSPORT MEMBRANE INCLUDING METAL COMPLEX

(71) Applicant: BATTELLE ENERGY ALLIANCE, LLC, Idaho Falls, ID (US)

(72) Inventors: Aaron D. Wilson, Idaho Falls, ID (US); John R. Klaehn, Idaho Falls, ID (US); Alan K. Wertsching, Idaho Falls, ID (US); Christopher J. Orme, Firth, ID (US); Eric S. Peterson, Idaho Falls, ID (US)

(73) Assignee: BATTELLE ENERGY ALLIANCE, LLC, Idaho Falls, ID (US)

(21) Appl. No.: 13/653,234

(22) Filed: Oct. 16, 2012

(51) Int. Cl. C08K 5/50; C07F 1/10; C07C 7/144; C08G 77/398; C08F 110/06; C08F 110/02; C08G 69/48; C08G 75/20; C08G 64/00; C08G 73/04; C07F 1/12; C08G 79/06

(52) U.S. Cl. 524/116; 556/21; 585/818; 525/475; 525/538; 525/333.7; 525/420; 525/535; 525/461; 525/540

(57) ABSTRACT

A membrane includes a metal or coordination complex that selectively interacts with one or more materials. The membrane can be used for facilitated transport separation of the materials. The metal complex can include any suitable metal center, but preferably includes a late transition metal. The metal complex can also include any suitable ligand, but preferably includes a triphosphacyclononane. The metal complex can be covalently linked to the membrane.

### STATEMENT REGARDING FEDERALLY SPONSORED RESEARCH OR DEVELOPMENT

This invention was made with government support under Contract No. DE-AC07-05-ID14517, awarded by the U.S. Department of Energy. The U.S. government has certain rights in the invention.

### BACKGROUND

The incorporation of metal ions into membranes is a problem at the border of membrane development and coordination chemistry, two well developed fields whose intersection is nascent. There are two historic methods of incorporating metal ions into membranes. The first is to add “free” metal ion salts to the stock solution used to produce the membrane. The second is a form of salt metathesis for membranes that already have an ionic content in which the “free” metal ion displaces protons, alkali metals, or alkaline earth metals normally present in the membrane. Unfortunately, there are at least two major problems with these methods. Neither of these methods controls the first or primary coordination sphere of the metal center and there is nothing to guarantee that the metal ions are sufficiently anchored.

For condensed phase materials the idea of a “free” naked metal ion is inappropriate. Established coordination chemistry indicates that the metal ion will be part of a crystal lattice, supported by discrete ligands, or part of a primary solvation sphere where the solvent acts as the primary ligand.

Without an effective chelator to anchor the metal ion to the membrane there is no guarantee that the ion will remain bound to the membrane. Two main factors that influence the effectiveness of a chelator are the kinetic stability of the chelate-metal bond under the relevant conditions and the thermodynamic strength of the bond with the former believed to be the more significant factor. Thus, metal ions used with membranes should be anchored with chelators that are kinetically stable under the relevant operating conditions.

The reactivity of a metal center is determined by three primary interdependent features: the elemental identity of the metal, the metal's oxidation state, and the metal's primary coordination sphere. The primary coordination sphere of a metal center strongly influences its binding strength to a substrate as well the metal center redox potentials and stability of various oxidation states. Thus the stability of the metal ion to degradation through oxidation or reduction is in part controlled by the ion's first coordination sphere.

Membranes such as these can be used to separate different materials through facilitated transport, which is the selective transport through the membrane of one material or class of materials over other materials present. The transport is facilitated by the metal center present in the membrane. Facilitated transport can improve separation any place a metal center selectively interacts with a component of the feedstock.

The most commonly known facilitated transport (active transport) is found in biological systems. Living cells have biomembranes that protect the cell from the external world, while still allowing the cell to interact with it. For example, the living cell includes specific channels located in the cell membrane that actively transport materials into the cell and reject undesirable materials out of the cell.

Facilitated gas separations with polymer membranes can use a similar approach to a living cell. A gas in the feedstock is selectively transported through the membrane over other gases by forming a complex in the membrane, which actively assists the gas through the membrane.

Efforts to advance the use of transition-metal center ions for use in facilitated transport agents cannot afford to ignore the role of a metal center's primary coordination sphere in regards to anchoring the ion to the membrane and moderating its activity as a transport agent. Solving these overlapping problems is the next step in taking facilitated transport from a nascent field of study to an applied technology.

One area of particular interest is the use of facilitated transport to separate olefins (double bonds, unsaturated hydrocarbons) and paraffins (aliphatic, saturated hydrocarbons). Olefin production in the USA is a multibillion dollar industry that uses many different refinery processes to generate olefins. They are used as organic feedstocks for the production of polymers used to make plastics. The refinery processes produce both olefins and paraffins at the same time and separating them is not simple.

The current state of the art for separating olefins and paraffins utilizes expensive cryogenic distillation. The primary problem with olefin/paraffin separations is that there is only one chemical handle that can be exploited for this separation, which is the double bond(s) on the olefin. Here, certain metal ions are known to have the ability to bind with olefins. Metal ions can complex with the olefins and provide facilitated transport of the olefin (as a gas or vapor) through a polymer membrane over paraffins.

Olefin facilitated transport has been intensely examined for a long time, and the best olefin/paraffin separations have been achieved using Cu(I) and Ag(I) ions in various polymer membranes. However, the disadvantage of these two metal ions is that they tend to reduce or oxidize to other stable oxidation states, thus becoming unusable in these separation processes.

### SUMMARY

A membrane includes a metal or coordination complex that provides facilitated transport of certain materials through the membrane. The metal complex can separate active materials—i.e., any material that interacts with the metal complex—from a feed stream by selectively transporting them across the membrane. Preferably, the metal complex is covalently bonded to the membrane.

The metal center of the metal complex can interact with one compound over other compounds. The metal center may selectively interact with almost any chemically unique lone pair or double bond, including olefins, O₂, CO, CO₂, NH₃, H₂O, etc. The selective interaction with the metal center may be used to separate these materials through processes such as facilitated transport. For example, the membrane may be used for facilitated transport separation of the following materials: olefin/paraffin, O₂/N₂ (air), NH₃/(N₂+H₂), CO₂/Mixture, CO/Mixture, H₂/Mixture, H₂O/Mixture, and the like.

The metal complex can include any suitable metal as the metal center. For example, the metal center can include early and late transition metals and main group metals. Late transition metals are preferred due to their oxygen tolerance.

The metal complex can also include any suitable ligand or combination of ligands. Preferably, the metal complex includes a macrocyclic ligand such as a heteromacrocyclic triphosphine or macrocyclic cyclononane. In one embodiment, the macrocyclic cyclononane includes phosphorus. Preferably, the ligand is a triphosphacyclononane.

In one embodiment, the metal complex has the following structural and chemical features, which are believed to produce more effective transport agents: (a) a late-transition metal center with a tolerance of oxygen-rich molecules, (b) a robust ligand structure that produces metal complexes that resists kinetic dissociation, (c) ability to complex with as many open coordination sites as possible to facilitate interactive transport, and (d) a ligand that binds securely to the membrane through a covalent bond or linkage of similar strength.

One example of a metal complex that has the features mentioned above has the general form of [M([9]-aneP₃R₃)(L)ₘ]ⁿ⁺. These metal complexes are particularly effective when the metal center is a transition metal and especially a late transition metal. The [9]-aneP₃R₃ ligand can retain late-transition metals under a wide range of environments.

The [9]-aneP₃R₃ ligand is 1,4,7-Tri(R)-1,4,7-triphosphacyclononane. The [9]-aneP₃R₃ ligand is a soft ligand that supports late-transition metals in a reduced state with an excellent complex geometry. This makes it an effective support for reduction catalysts.

In one embodiment, the membrane is used for facilitated transport separation of olefins and paraffins having two to six carbon atoms—i.e., C2 to C6. The membrane includes metal complexes having metal centers of Cu(I) or Ag(I). The ligand stabilizes the metal ions by lessening their susceptibility to reduction or oxidation while retaining an active binding site for the olefin.

### DRAWINGS

FIG. 1 shows the structure of two embodiments of a metal complex that can be linked to a membrane. The metal complexes have the formula M([9]-aneP₃Rₖ)(L)ₘ]ⁿ⁺ where k is 2 or 3.

FIG. 2 shows one embodiment of a method to synthesize [9]-aneP₃R₃ by reacting dilithiated bis(2-Rphosphidoethyl)Rphosphine with 1,2-dichloroethane.

FIG. 3 shows the synthesis of a derivative of [9]-aneP₃R₃ including a hydroxyl functional group that reacts with an acyl halide to covalently link the [9]-aneP₃R₃ ring to a membrane or a chemical group to enhance membrane retention.

FIG. 4 shows the synthesis of a derivative of [9]-aneP₃R₃ including a carboxyl group that reacts with n-hydroxymethyl phthalimide to form an activated phthalimide that can react with an amine in the membrane to covalently link the [9]-aneP₃R₃ ring to the membrane or a chemical group to enhance membrane retention.

FIG. 5 shows the synthesis of a derivative of [9]-aneP₃R₃ including a vinyl group that can co-polymerize to directly incorporate the [9]-aneP₃R₃ ring into the membrane or a chemical group or polymer to enhance membrane retention.

FIG. 6 shows the synthesis of a derivative of [9]-aneP₃R₃ including a protected alcohol as a phosphine substituent group that can be used to link the [9]-aneP₃R₃ ring into the membrane or a chemical group to enhance membrane retention.

### DETAILED DESCRIPTION

A membrane is disclosed that can be use for facilitated transport separation of a feed stream. The membrane includes one or more metal complexes having a metal center that selectively interacts with one or more materials in the feed stream. The membrane can be used to separate almost any material that interacts with the metal complex—i.e., an active material—from materials that do not interact—i.e., an inactive or non-participating material.

The metal complex can selectively interact with almost every chemically unique lone pair or double bond, including olefins, O₂, CO, CO₂, NH₃, H₂O, etc. The membrane can be used to separate olefin/paraffin, O₂/N₂ (air), NH₃/(N₂+H₂), CO₂/Mixture, CO/Mixture, H₂/Mixture, H₂O/Mixture. The olefin/paraffin separation is described in detail to illustrate one example of a suitable use for the membrane. However, it should be appreciated that the membrane can be used to separate any material that selectively interacts with the metal complex.

The metal complex interacts with the active material and facilitates transport of the active material across the membrane. The active material is released on the opposite side of the membrane and collected. The interaction is reversible to allow the active material to be released after it moves through the membrane.

The flux rate of the active material is determined by the transport rate of the metal complex through the membrane. The flux rate of the active material is a function of the flux and concentration of the metal complex. A number of other factors, such as the thickness of the membrane, may also affect the flux rate of the active material.

The reactivity of the metal complex is influenced by a number of factors, but especially by the following factors: the elemental identity of the metal, the metal's oxidation state, and the metal's primary coordination sphere. The reactivity can be adjusted to account for the specific active material being separated, process conditions, and so forth.

The metal complex may include any suitable metal center, including early and late transition metals and main group metals. A late-transition metal center is preferred due its oxygen tolerance. Early-transition metal centers and main group metal centers often react irreversibly with oxygen and organic molecules containing alcohols or even ketones and ethers thereby rendering them unusable. Such sensitivity can be a serious draw back in separation environments that deal with unpurified feedstocks. In contrast late-transition metal centers can tolerate the presence of oxygen containing molecules and a robust array of operating conditions.

In one embodiment, the metal center includes any ion of a late-transition metal i.e., Fe, Co, Ni, Cu, Zn, Ru, Rh, Pd, Ag, Cd, Os, Ir, Pt, Au, Hg. Examples of ions that may be especially suitable as the metal center include Cu(I), Ag(I).

The first or primary coordination sphere of the metal center strongly influences its binding strength as well as the metal center redox potentials and stability of various oxidation states. Thus the susceptibility of the metal ion to degradation through oxidation or reduction is in part controlled by the ion's first coordination sphere.

The first coordination sphere may include any suitable ligand or combination of ligands. Preferably, the metal complex includes a macrocyclic ligand. In one embodiment, the macrocyclic ligand includes phosphorus. For example, the macrocyclic ligand may be a heteromacrocyclic triphosphine. In another embodiment the macrocyclic ligand includes a cyclononane ring. Preferably, the macrocyclic ligand includes a triphosphine cyclononane.

In one embodiment, the metal complex includes the [9]-aneP₃R₃ ligand. It is not only attractive for its chemical properties but also for its novelty. The [9]-aneP₃R₃ ligand is a relatively new ligand platform which has very different overall coordination chemistry and reactivity than other well-known ligands such as diphosphines, thio crowns, and the like.

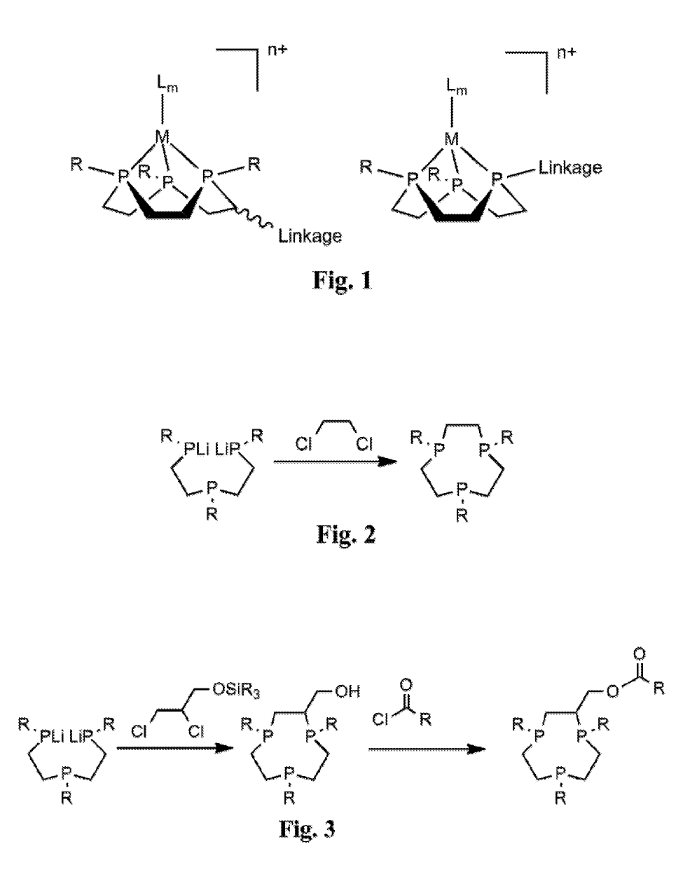

The [9]-aneP₃R₃ ligand is a robust ligand with high thermodynamic binding, rapid complex formation, and minimal kinetic lability. It is a tridentate ligand having three soft phosphine donors which are especially suited for coordination with late-transition metal centers in all oxidation states. The [9]-aneP₃R₃ ligand forms a ring around part of the metal center as shown in FIG. 1. The ring effect enhances the kinetic stability of the metal complex over comparable branched polydentate ligands which can readily dissociate through the sequential release of chelate arms.

The [9]-aneP₃R₃ ligand forms a robust metal complex while still retaining accessible open coordination sites suitable for active transport or catalysis. As a tridentate facial ligand, [9]-aneP₃R₃ supports native octahedral, tetrahedral, most five-coordinate geometries, most three-coordinate geometries, and most two-coordinate geometries while retaining one to three open coordination sites for substrate interactions and transport. The triphosphacyclononane ligand has the ability to facially cap the metal center and thereby make it kinetically and thermally stable, while creating labile positions trans to the phosphorus coordination sites.

The [9]-aneP₃R₃ ligand includes phosphines which form stable complexes in a variety of environments such as humid, acidic, basic, and high temperature environments. The [9]-aneP₃R₃ ligand retains the metal center even when the metal center is reduced to a neutral state. This prevents the metal centers from sintering through autocatalytic propagation. If too many of the metal centers are reduced, it may affect the function of the membrane. However, the membrane can be regenerated by exposing it to an oxidizing agent.

The [9]-aneP₃R₃ ligand features a minimal amount of steric bulk and crowding. As a tridentate ligand, [9]-aneP₃R₃ has three total substituents (R) while bidentate phosphine ligands generally have four substituents. Furthermore, these three substituents as well as the [9]-aneP₃R₃ ligand's back bone ring are positioned away from the open coordination sites leaving them accessible for active transport or catalysis. For example, the geometry of Cu(I) or Ag(I) metal complexes leave open the active d-orbitals to interact with the target material.

The three M-P bonds formed by the [9]-aneP₃R₃ ligand and the metal center are geometrically oriented to avoid orbital competition through trans influence. In addition, the strength of the three M-P bonds increases the lability of the “open” coordination sites through a trans effect. This lability prevents the open sites intended for transport from becoming irreversibly occupied by solvent, substrate, or other poisonings.

The [9]-aneP₃R₃ ligand may include any suitable substituents. For example, R can be any suitable hydrocarbon. Preferably, each R is the same material. However, it should be appreciated that in some embodiments each R may be a different material. In one embodiment, R is independently alkyl, aryl; such as methyl, ethyl, n-propyl, iso-propyl, n-butyl, iso-butyl, t-butyl, neo-pentyl, cyclopentyl, cyclohexyl, n-octyl, hydroxylmethyl, CH₂N(CH₃)₂, CH₂N(R)₂, 2-furyl, phenyl, (m, o, and p)-tolyl, (m, o, and p)-methoxyphenyl, (m, o, and p)-sulfonato, (m, o, and p)-halophenyl, (m, o, and p)-trifluoromethylphenyl, and pentafluorophenyl among others. In another embodiment, the [9]-aneP₃R₃ ligand is 1,4,7 triphenyl-1,4,7-triphosphacyclononane.

The [9]-aneP₃R₃ ligand can be readily modified with a variety of functional groups that covalently link to the chemical structure of the membrane or a chemical component that enhances membrane retention. A number of these derivatives are shown in FIGS. 3-6 and described in greater detail below. This linkage ensures that the metal complex remains attached to the membrane even if the conditions would normally solvate the metal complex.

The [9]-aneP₃R₃ ligand forms metals complexes that have the general form of [M([9]-aneP₃R₃)(L)ₘ]ⁿ⁺. These metal complexes are particularly effective when the metal center is a transition metal and especially a late transition metal. The [9]-aneP₃R₃ ligand can retain late-transition metals under a wide range of environments.

The substituent L in the metal complex can be any suitable material. For example, L can be halo, hydroxo, aqua, carbonyl, substituted phosphines, amino, substituted amines, pyridine, acetonitrile, dimethyl sulfoxide, tetrahydrofuran, and other solvents among other ligands.

The metal complexes can be prepared using any suitable process. The first step is to prepare the ligand. The [9]-aneP₃R₃ ligand can be synthesized using the method described in Lowry et al., Synthesis of 1,4,7-Triphenyl-1,4,7-triphosphacyclononane: The First Metal-Free Synthesis of a [9]-aneP₃R₃ ring, Inorg. Chem. 2010, 49, 4732-4734, which is incorporated by reference herein in its entirety.

The process includes reacting lithium bis(2-Rphosphidoethyl)Rphosphine with 1,2-dihaloethane (e.g., 1,2-dichloroethane or 1,2-dibromoethane) at low concentration and high temperature. The reaction conditions produce the intramolecular ring-closed product over intermolecular oligomeric and polymeric materials. The [9]-aneP₃R₃ rings are separated from the reaction mixture through solvent extraction. One example of this reaction is illustrated in FIG. 2 where the halogenated ethane is 1,2-dibromoethane.

FIGS. 3-6 show variations of the reaction in FIG. 2 that produce derivatives of [9]-aneP₃R₃ having functional groups that can covalently link to various membranes or a chemical component that enhances membrane retention. FIG. 3 shows a derivative of [9]-aneP₃R₃ that includes a hydroxyl group that can be used to link the [9]-aneP₃R₃ ring to a wide variety of membranes or a chemical component that enhances membrane retention. For example, the hydroxyl group can react with an acyl halide that is part of the membrane to covalently link the [9]-aneP₃R₃ ring to the membrane or a chemical component that enhances membrane retention.

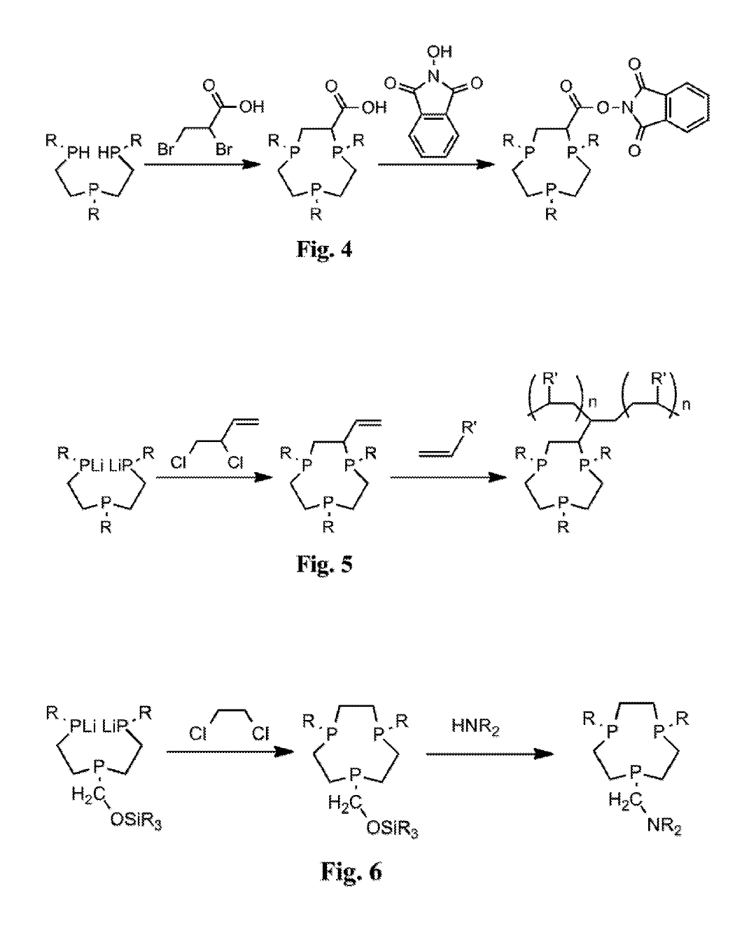

FIG. 4 shows a derivative of [9]-aneP₃R₃ that includes a carbonyl group that can be used to link the [9]-aneP₃R₃ ring to various membranes or a chemical component that enhances membrane retention. For example, the carbonyl group can react with n-hydroxymethyl phthalimide to form an activated phthalimide as shown in FIG. 4. The activated phthalimide can react with an amine in the membrane to covalently link the [9]-aneP₃R₃ ring to the membrane or a chemical component that enhances membrane retention.

FIG. 5 shows a derivative of [9]-aneP₃R₃ that includes a vinyl group that can be used to directly co-polymerize the [9]-aneP₃R₃ ring with the polymeric material that forms the membrane. In this way, the [9]-aneP₃R₃ ring is covalently linked to the polymeric membrane or a chemical component that enhances membrane retention.

The metal complex is formed by reacting the ligand with the selected metal ion. For example, a solution of [9]-aneP₃R₃ is reacted with Ag(BF₄) to form fac-Ag([9]-aneP₃R₃)(L)₃(BF₄) metal complex.

The [9]-aneP₃R₃ ligand can also be synthesized using other processes such as template synthesis. Examples of suitable template synthesis processes are described in Edwards, et al., Template Synthesis of 1,4,7-Triphosphacyclononanes, J. Am. Chem. Soc. 2006, 128, 3818-30 and Edwards et al., Template Synthesis of the First 1,4,7-Triphosphacyclononane Derivatives, Angew. Chem. Int. Ed. 2000, 39, No. 16, 2922-24 both of which are incorporated by reference herein in their entirety.

Although templated macrocyclic ligands are acceptable, there are still challenges associated with these materials. One challenge is that the templated synthesis of macrocyclic triphosphines produces very stable metal complexes that tend to be poor catalysts and lack the reactivity necessary to interact with the active material. The templated complexes are also not good synthetic starting points to make other metal complexes because their stability makes it difficult to remove macrocyclic triphosphines from the template for use with a different metal center. While in principle it may be possible to remove a macrocyclic triphosphine from its template the reaction yields and atom economy are so poor that such transfers are often not practical.

The metal complexes may be used with any suitable membrane. In one embodiment, the membrane includes polymeric material. Preferably, the membrane is capable of covalently bonding with the metal complex. Examples of suitable membranes include (1) rubbery/soft types of polymers, like polysiloxanes, polyphosphazenes, polyethylene, polypropylene and polyalkyl oxides (ethyl and propyl), (2) glassy polymers, like polyimides (such as Matrimid 5218), polyamides (such as nylon), and polybenzazoles (such as PBI) along with polysulfones, PET-types, polycarbonates, PEEK, PEI, etc., (3) inorganic metal-oxide support materials, like silicates, aluminates, titantia, etc., and (4) metal and porous metal supports.

Although it is preferable to covalently link the metal complexes to the membrane structure, it should be appreciated that the metal complex may be incorporated into the membrane in other ways. For example, during production of the membrane, the metal complex may be intermixed with the material that forms the membrane. Although the metal complex is not chemically bound to the membrane, it is physically present in the membrane.

In one embodiment, the membrane is used to separate olefins and paraffins. The metal complex is [M([9]-aneP₃R₃)(L)ₘ]ⁿ⁺ where M is Cu(I) or Ag(I). As described above, the metal complex is stable so that the metal ions are less likely to reduce or oxidize and become unusable. The membrane may be any of those listed above.

The terms recited in the claims should be given their ordinary and customary meaning as determined by reference to relevant entries (e.g., definition of “plane” as a carpenter's tool would not be relevant to the use of the term “plane” when used to refer to an airplane, etc.) in dictionaries (e.g., widely used general reference dictionaries and/or relevant technical dictionaries), commonly understood meanings by those in the art, etc., with the understanding that the broadest meaning imparted by any one or combination of these sources should be given to the claim terms (e.g., two or more relevant dictionary entries should be combined to provide the broadest meaning of the combination of entries, etc.) subject only to the following exceptions: (a) if a term is used herein in a manner more expansive than its ordinary and customary meaning, the term should be given its ordinary and customary meaning plus the additional expansive meaning, or (b) if a term has been explicitly defined to have a different meaning by reciting the term followed by the phrase “as used herein shall mean” or similar language (e.g., “herein this term means,” “as defined herein,” “for the purposes of this disclosure [the term] shall mean,” etc.).

References to specific examples, use of “i.e.,” use of the word “invention,” and so forth, are not meant to invoke exception (b) or otherwise restrict the scope of the recited claim terms. Other than situations where exception (b) applies, nothing contained herein should be considered a disclaimer or disavowal of claim scope. The subject matter recited in the claims is not coextensive with and should not be interpreted to be coextensive with any particular embodiment, feature, or combination of features shown herein. This is true even if only a single embodiment of the particular feature or combination of features is illustrated and described herein. Thus, the appended claims should be read to be given their broadest interpretation in view of the prior art and the ordinary meaning of the claim terms.

As used herein (i.e., in the claims and the specification), articles such as “the,” “a,” and “an” can connote the singular or plural. Also, the word “or” when used without a preceding “either” (or other similar language indicating that “or” is unequivocally meant to be exclusive—e.g., only one of x or y, etc.) shall be interpreted to be inclusive (e.g., “x or y” means one or both x or y). Likewise, the term “and/or” shall also be interpreted to be inclusive (e.g., “x and/or y” means one or both x or y). In situations where “and/or” or “or” are used as a conjunction for a group of three or more items, the group should be interpreted to include one item alone, all of the items together, or any combination or number of the items. Moreover, terms used in the specification and claims such as have, having, include, and including should be construed to be synonymous with the terms comprise and comprising.

Unless otherwise indicated, all numbers or expressions, such as those expressing dimensions, physical characteristics, etc. used in the specification (other than the claims) are understood as modified in all instances by the term “approximately.” At the very least, and not as an attempt to limit the application of the doctrine of equivalents to the claims, each numerical parameter recited in the specification or claims which is modified by the term “approximately” should at least be construed in light of the number of recited significant digits and by applying ordinary rounding techniques.

All ranges disclosed herein are to be understood to encompass and provide support for claims that recite any and all subranges or any and all individual values subsumed therein. For example, a stated range of 1 to 10 should be considered to include and provide support for claims that recite any and all subranges or individual values that are between and/or inclusive of the minimum value of 1 and the maximum value of 10; that is, all subranges beginning with a minimum value of 1 or more and ending with a maximum value of 10 or less (e.g., 5.5 to 10, 2.34 to 3.56, and so forth) or any values from 1 to 10 (e.g., 3, 5.8, 9.9994, and so forth).

What is claimed is:

1. A membrane comprising a metal complex including triphosphacyclononane.

2. The membrane of claim 1 wherein the metal complex is covalently bonded to the membrane or a chemical component that enhances membrane retention.

3. The membrane of claim 1 wherein the metal complex includes a late transition metal center.

4. The membrane of claim 1 wherein the metal complex includes Cu(I) or Ag(I).

5. The membrane of claim 1 wherein the membrane includes polymeric material.

6. The membrane of claim 5 wherein the polymeric material and the metal complex are part of a copolymer.

7. The membrane of claim 1 wherein the metal complex includes 1,4,7-triR-1,4,7-triphosphacyclononane or a derivative of 1,4,7-triR-1,4,7-triphosphacyclononane where R is, independently, methyl, ethyl, n-propyl, iso-propyl, n-butyl, iso-butyl, t-butyl, neo-pentyl, cyclopentyl, cyclohexyl, n-octyl, hydroxylmethyl, CH₂N(CH₃)₂, CH₂N(R)₂, 2-furyl, phenyl, (m, o, and p)-tolyl, (m, o, and p)-methoxyphenyl, (m, o, and p)-sulfonato, (m, o, and p)-halophenyl, (m, o, and p)-trifluoromethylphenyl, or pentafluorophenyl.

8. A membrane comprising:
a membrane structure; and
a metal complex including triphosphacyclononane, the metal complex including a late transition metal center;
wherein the metal complex is covalently bonded to the membrane structure.

9. The membrane of claim 8 wherein the metal complex includes Cu(I) or Ag(I).

10. The membrane of claim 8 wherein the membrane includes polymeric material.

11. The membrane of claim 10 wherein the polymeric material and the metal complex are part of a copolymer.

12. The membrane of claim 8 wherein the metal complex includes 1,4,7-triR-1,4,7-triphosphacyclononane or a derivative of 1,4,7-triR-1,4,7-triphosphacyclononane where R is, independently, methyl, ethyl, n-propyl, iso-propyl, n-butyl, iso-butyl, t-butyl, neo-pentyl, cyclopentyl, cyclohexyl, n-octyl, hydroxylmethyl, CH₂N(CH₃)₂, CH₂N(R)₂, 2-furyl, phenyl, (m, o, and p)-tolyl, (m, o, and p)-methoxyphenyl, (m, o, and p)-sulfonato, (m, o, and p)-halophenyl, (m, o, and p)-trifluoromethylphenyl, or pentafluorophenyl.

13. A separation method comprising:
separating a feed stream with a membrane including a metal complex;
wherein the metal complex includes triphosphacyclononane; and
wherein the metal complex selectively interacts with and facilitates the transport of one or more materials in the feed stream through the membrane.

14. The separation method of claim 13 wherein the feed stream includes olefins and paraffins and the metal complex selectively interacts with the olefins to separate the olefins and paraffins.

15. The separation method of claim 13 wherein the metal complex is covalently bonded to the membrane.

16. The separation method of claim 13 wherein the metal complex includes a late transition metal center.

17. The separation method of claim 13 wherein the metal complex includes Cu(I) or Ag(I).

18. The separation method of claim 13 wherein the membrane includes polymeric material.

19. The separation method of claim 18 wherein the polymeric material and the metal complex are part of a copolymer.

20. The separation method of claim 13 wherein the metal complex includes 1,4,7-triR-1,4,7-triphosphacyclononane or a derivative of 1,4,7-triR-1,4,7-triphosphacyclononane where R is, independently, methyl, ethyl, n-propyl, iso-propyl, n-butyl, iso-butyl, t-butyl, neo-pentyl, cyclopentyl, cyclohexyl, n-octyl, hydroxylmethyl, CH₂N(CH₃)₂, CH₂N(R)₂, 2-furyl, phenyl, (m, o, and p)-tolyl, (m, o, and p)-methoxyphenyl, (m, o, and p)-sulfonato, (m, o, and p)-halophenyl, (m, o, and p)-trifluoromethylphenyl, or pentafluorophenyl.
